(19) United States
(12) Patent Application Publication
(10) Pub. No.: US 2022/0138998 A1
(43) Pub. Date: May 5, 2022

(54) SYSTEM AND METHOD FOR AUGMENTED REALITY VISUALIZATION OF BIOMEDICAL IMAGING DATA

(71) Applicant: Vasoptic Medical Inc., Baltimore, MD (US)

(72) Inventors: Abhishek Rege, Ellicott City, MD (US); Jayanth Kandukuri, Ellicott City, MD (US); Aseem Jain, Baltimore City, MD (US); Aleksandr Smirnov, Gainesville, VA (US); M. Jason Brooke, Baltimore, MD (US)

(21) Appl. No.: 17/427,802

(22) PCT Filed: Feb. 3, 2020
(86) PCT No.: PCT/US2020/016312

§ 371 Date: Aug. 2, 2021

Related U.S. Application Data
(60) Provisional application No. 62/800,920, filed on Feb. 4, 2019.

(51) Int. Cl. G06T 11/00; G02B 21/00; G02B 27/01; G02B 21/08; G02B 27/48; G02B 21/36

(52) U.S. Cl. CPC G06T 11/00; G02B 21/0012; G02B 27/0101; G02B 21/08; G02B 2027/014; G02B 21/367; G02B 2027/0141; G02B 2027/0138; G02B 27/48

(57) ABSTRACT

Augmented reality eyepiece systems for surgical microscopes and associated methods are disclosed. An example augmented reality eyepiece can include a processor configured to generate a signal based on image data pertaining to a field of view of a surgical microscope and an eyepiece configured to integrate with the surgical microscope. The eyepiece can include an image generation module configured to generate an image based on the data signal, an image combiner configured to combine the image generated by the image generation module with light received from the field of view to create a combined image, and visualization optics configured to present the combined image to an eye of a user of the surgical microscope.

### RELATED APPLICATIONS

This application claims the benefit of U.S. Provisional Application No. 62/800,920, filed Feb. 4, 2019, the entirety of which is incorporated herein by reference.

### BACKGROUND

Surgical optics can be used for remote and/or magnified viewing of a field of view or a patient or subject. The surgical optics can take various forms such as microscopes, endoscopes, laparoscopes, loupes, goggles, etc. A surgeon or other operator may benefit from additional information other than the view provided directly by the surgical optics. For many surgical procedures, however, it may be inconvenient or risky for the surgeon to look away from the view of the optics, or from the field of view. Also, histopathologists often prefer direct sample observation but would like to have machine assistance during examination of multiple sections at large fields of view afforded by modern optics. Same can be said about ophthalmologists examining eye fundus with specialized cameras during routine checkups and while diagnosing adverse events.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. Subsystems are normally surrounded by dashed lines, and some of them may be optional. Visual information or optical signal travel is indicated by means of dash-dotted arrows. Optional procedures and data flow are indicated with dashed arrows or elements in methods illustrations by flowcharts. In the drawings:

FIG. 1 is a block diagram illustrating an example embodiment of a system for real-time examination of particulate flow in a target tissue;

FIGS. 2A and 2B illustrate example embodiments of Augmented Reality displays in context of surgical stereomicroscopes utilizing one (monoscopic) or both (for stereo effect) eyepieces;

FIG. 3 illustrates an example embodiment of a laser speckle contrast/ICG-VA imaging system in context of Augmented Reality System, based on a remote color display panels or an in-line eye-piece microdisplay (monoscopic);

FIG. 4 is a flowchart illustrating overview of an example embodiment of a method of operation of a real-time multi-modality Augmented reality (AR) system;

FIGS. 5A and 5B show a flowchart depicting an example embodiment of a method for rapid examination of particulate flow using laser speckle contrast imaging (LSCI) object in context of other modalities;

FIGS. 6A and 6B show a flowchart of depicting an example embodiment of a method for rapid examination of a target object by employing fluorescence, l phosphorescence, luminescence, harmonic generation, ultrasound, acousto-optical and opto-acoustic, spontaneous or coherent scattering-based imaging in context of LSCI and other modalities; and

FIGS. 7A and 6B show a flowchart depicting an example embodiment of a method for multi-spectral or multi-wavelength imaging of target object in context of LSCI and other modalities.

### DETAILED DESCRIPTION OF THE EMBODIMENTS

The following detailed description of the present subject matter refers to the accompanying drawings that show, by way of illustration, specific aspects and embodiments in which the present subject matter may be practiced. These embodiments are described in sufficient detail to enable those skilled in the art to practice the present subject matter. The subject technology can assume various embodiments that are suitable to its specific applications.

The present disclosure relates generally to systems and methods for presentation of static or dynamic information within an image projection subsystem, in various types of telescopic, macroscopic, microscopic, laparoscopic, and endoscopic imaging applications.

This disclosure pertains to providing the user of an imaging system, for example a microscope or a telescope, the ability to view augmented information pertaining to the field of view. This disclosure describes systems and methods to optically overlay recently or concurrently acquired and processed or stored data on to a field of view of an imaging system. An embodiment of such a system may be described as a digital eyepiece, that is, an eyepiece with an inbuilt display module receiving electronic data that can provide visualization of the microscope's field of view augmented with a static or dynamic textual, numerical, graphical, or image rendition of the electronic data. An embodiment of such a system may be useful as a diagnostic, pre-operative and especially intraoperative tool during surgery, where the digital eyepiece receives calculated blood flow data from the surgical field of view in real-time and with minimal delay, and overlays a pseudo-color rendition of blood flow index onto the original visual field, thereby permitting the surgeon or histopathologist to instantaneously visualize this critical information without looking away from the eyepiece of the operation microscope. The system and method may be useful to visualize data obtained using other imaging modalities in other professional and recreational activities, too, and due to its real-time nature, can be classified as a type of Augmented Reality (AR).

In our personal and professional life we sometimes encounter situations where their direct visual perception, however aided or enhanced by means of an optical system, be it a “magnifier” or an “intensifier”, needs an additional augmentation with information not readily available or detected by our natural senses or neural circuitries. Such relevant information, however, may be retrieved from an external storage or derived in real time by means of artificial signal or image registration and processing systems; for example, machine vision devices. And when presented in a timely fashion in context of a current situation a substantially more enhanced or informed version of reality thus delivered to user's consciousness. Recently termed as Augmented Reality (AR), such aid to our senses and brain processing capacity promises tremendous improvement in a person's situational awareness, decision making process and reliability of predicting the final outcome in a dynamic situation.

One example of such an area of activity, often in need of additional critical information delivered to a person's field of perception, is medical surgery where procedure success or failure usually depends on real-time response of a practitioner to the actively developing patient's state and the situation. Nor normally perceptible, but routinely critical to the patient, are characteristics of blood flow (pressure, speed and direction and their dynamics) within his or her cardiovascular system. The cardiovascular system is the fundamental mechanism by which human and animal organisms provide nutrient supply to, and remove waste products from, tissues, organs, and organ systems to maintain their homeostasis, viability, integrity, and functionality. Anatomical characteristics of blood vessels (e.g., size, structure, orientation, location, quantity, distribution, and type) are specific to each biological system, tissue, organ, or organ system. Many pathologies manifest as changes in these anatomical characteristics and are also accompanied by changes in vascular physiology (e.g., velocity or flow rates of blood within an individual vessel, group of vessels, or network of vessels; distribution of blood flow within a group or network of connected or independent vessels, including regions of vascular profusion and non-perfusion; and vessel compliance, contractility, and proliferation). For example, diabetic retinopathy (DR) is a vision-threatening complication of diabetes that manifests as progressive narrowing of arteriolar caliber until occlusion occurs. Vessel occlusion is typically followed by vessel proliferation (i.e., angiogenesis), which results in increased vision loss and progression toward blindness. Numerous other diseases and conditions involve pathologies or symptoms that manifest in blood vessel anatomy or physiology. As another example, various dermatological diseases and conditions, including melanoma, diabetic foot ulcers, skin lesions, wounds, and burns, involve injury to or pathophysiology of the vasculature. In neurosurgery and tissue or organ reconstruction, temporarily disrupting and then reestablishing normal blood flow to a particular area is a critical step in success of procedures, often performed under an operation microscope.

These anatomical and physiological characteristics are important for the quality application of existing and development of novel diagnostics and procedures for the advancement of standard of care for patients (human and animal). By evaluating the anatomical and physiological characteristics of the vasculature (directly or indirectly, quantitatively or qualitatively), a scientist, clinician, or veterinarian can begin to understand the viability, integrity, and functionality of the biological system, tissue, organ, or whole organism being studied. Depending on the specific condition being studied, important markers may manifest as acute or long-term alterations in blood flow, temperature, pressure or other anatomical and physiological characteristics of the vasculature. For example, anatomical and physiological information, in either absolute terms or as relative changes, may be used as a mechanism for evaluating grade and character of brain aneurisms and inform of potential blood flow management and treatment options, among other things. Likewise, almost all types of tumors are accompanied by vascular changes to the cancerous tissue; tumor angiogenesis and increased blood flow is often observed in cancerous tissue due to increased metabolic demand of the tumor cells. Similar vascular changes are associated with healing of injuries, including wounds and burns, where self-regulated angiogenesis serves a critical role in the healing process. Hence, anatomical and physiological information may assist a clinician or veterinarian in the monitoring and assessment of healing after a severe burn, recovery of an incision site, or the effect of a therapeutic agent or other type of therapy (e.g., skin graft or negative pressure therapy) in the treatment of a wound or diabetic foot ulcer.

As already mentioned above, monitoring and assessment of anatomical and physiological information can be critically important for surgical procedures. The imaging of blood vessels, for example, can serve as a basis for establishing landmarks during surgery. During brain surgery, when a craniotomy is performed, the brain often moves within the intracranial cavity due to the release of intracranial pressure, making it difficult for surgeons to use preoperatively obtained images of the brain for anatomical landmarks. In such situations, anatomical and physiological information may be used by the surgeon as vascular markers for orientation and navigation purposes. Anatomical and physiological information also provides a surgeon with a preoperative, intraoperative, and postoperative mechanism for monitoring and assessment of the target tissue, organ, or an individual blood vessel within the surgical field.

The ability to quantify, visualize, and assess anatomical and physiological information in real-time or near-real-time can provide a surgeon or researcher with feedback to support diagnosis, treatment, and disease management decisions. An example of a case where real-time feedback regarding anatomical and physiological information is important is that of intraoperative monitoring during neurosurgery, or more specifically, cerebrovascular surgery. The availability of real-time blood flow assessment in the operating room (OR) allows the operating neurosurgeon to guide surgical procedures and receive immediate feedback on the effect of the specific intervention performed. In cerebrovascular neurosurgery, real-time blood flow assessment can be useful during aneurysm surgery to assess decreased perfusion in the feeder vessels as well as other proximal and distal vessels throughout the surgical procedure.

Likewise, rapid examination of vascular anatomy and physiology has significant utility in other clinical, veterinary, and research environments. For example, blood flow is often commensurate with the level of activity of a tissue and related organ or organ system. Hence, vascular imaging techniques that can provide rapid assessment of blood flow can be used for functional mapping of a tissue, organ, or organ system to, for example, evaluate a specific disease, activity, stimulus, or therapy in a clinical, veterinary, or research setting. To illustrate, when the somatosensory region of the brain is more active because of a stimulus to the hand, the blood flow to the somatosensory cortex increases and, at the micro-scale, the blood flow in the region of the most active neurons increases commensurately. As such, a scientist or clinician may employ one or more vascular imaging techniques to evaluate the physiological changes in the somatosensory cortex associated with the stimulus to the hand.

In addition, a number of imaging approaches exist to evaluate anatomical and physiological information of the tissue vasculature, its level of metabolism or stress condition. Magnetic resonance imaging (MRI), X-ray or computerized tomography (CT), ultrasonography, acousto-optical or opto-acoustic imaging, Doppler optical coherence tomography (D-OCT), laser speckle contrast imaging (LSCI), laser Doppler spectroscopy (LDS), polarization spectroscopy, multispectral reflectance spectroscopy, coherent Raman spectroscopy (CRS), spontaneous Raman spectroscopy, fluorescence angiography, fluorescence and phosphorescence lifetime imaging (FLIM and PLIM), and positron emission tomography (PET) are among a number of imaging techniques that offer quantitative and qualitative information about the vascular anatomy and tissue or organ physiology. Each technique offers unique features, not accessible by normal human faculties that may be more relevant to the real-time evaluation of a particular biological system, tissue, organ, or organ system or a specific disease or medical condition.

LSCI has particular relevance in the rapid, intraoperative examination of vascular anatomy and physiology. LSCI is an optical imaging technique that uses interference patterns (called speckles), which are formed when a camera captures photographs of a rough surface illuminated with coherent light (e.g., a laser), to estimate and map flow of various particulates in different types of enclosed spaces. If the rough surface comprises of moving particles, then the speckles corresponding to the moving particles cause a blurring effect during the exposure time over which the photograph is acquired under properly defined imaging conditions. The blurring can be mathematically quantified through the estimation of a quantity called laser speckle contrast (K), which is defined as the ratio of standard deviation to mean of pixel intensities in a given neighborhood of pixels. The neighborhood of pixels may be adjacent in the spatial (i.e., within the same photograph) or temporal (i.e., across sequentially acquired photographs) domains or a combination thereof. In the context of vascular imaging, LSCI quantifies the blurring of speckles caused by moving blood cells and other particulate such as lipid droplets, within the blood vessels of the illuminated region of interest (ROI) and can be used to analyze detailed anatomical information (which includes but is not limited to one or more of vessel diameter, vessel tortuosity, vessel density in the ROI or sub-region of the ROI, depth of a vessel in the tissue, length of a vessel, and type of blood vessel, e.g., its classification as artery or vein) and physiological information (which includes but is not limited to one or more of blood flow and changes thereof in the ROI or a sub-region of the ROI, blood flow in an individual blood vessel or group of individual blood vessels, and fractional distribution of blood flow in a network of connected or disconnected blood vessels).

While non-LSCI methods of intraoperative real-time blood flow assessment are currently used, no single method is considered adequate in all scenarios. For example, in the context of cerebrovascular surgery such as aneurysm surgery, imaging of small yet important vessels called perforators necessitates a high-resolution imaging technique for monitoring anatomical and physiological information, which is currently unavailable in the neurosurgical OR. The use of Indocyanine Green (ICG) Video angiography has been assessed for this purpose, but challenges still remain because of the potential for dye leakage from damaged blood vessels into surgical ROIs. Intraoperative (X-ray) angiography is currently considered the gold standard to assess vessel patency following a number of cerebrovascular procedures (e.g., aneurysm clipping and arteriovenous malformation, or AVM, obliteration). However, angiography does not provide real-time assessment during the actual performance of surgery. Furthermore, given the invasive nature of this technique, and despite advancements, the risk of complications is not eliminated, especially due to adverse side effects of contrast agents used. In AVM surgery, real-time blood flow assessment helps the surgeon better understand whether particular feeding vessels carry high flow or low flow, which could ultimately impact the manner in which those vessels are disconnected from the AVM (i.e., bipolar cautery versus clip ligation). Finally, in a disease such as Moyamoya, which may require direct vascular bypass, real-time flow assessment can be useful in identifying the preferred recipient vessels for the bypass as well as assessing the flow in that bypass and surrounding cortex once the anastomosis is completed.

The real-time assessment of blood flow may be helpful in other surgery fields that rely on vascular anastomoses as well, specifically plastic surgery, vascular surgery, and cardiothoracic surgery. Currently, technology such as the use of Doppler ultrasonography is used to confirm the patency of an anastomosis. However, real-time, quantitative imaging can add a tremendous benefit in assessing the adequacy of a bypass, revealing problems to the surgeon in real time to facilitate clinical decision making during surgery rather than postoperatively when either it is too late or the patient requires a repeat surgery.

LSCI has been used as a blood flow monitoring technique in the OR. LSCI has been considered for functional mapping in awake craniotomies to prevent damage to eloquent regions of the brain, to assess the surgical shunting of the superior temporal artery (STA) and the middle cerebral artery (MCA), and for intraoperative monitoring during neurosurgery. Until recently, these approaches had limitations of spatio-temporal resolution and availability of anatomical and physiological information on a real-time or near-real-time basis.

Of particular importance is the method for presentation and appearance of visual information obtained from LSCI and other modalities in the context of an OR environment. While projection of images and video sequences obtained, whether in monochromatic or pseudo-color format, to built-in or external displays is helpful in principle, in actual practice taking eye focus away from the operating field can amount to a disruptive distraction or inconvenience for a surgeon or his or her attendant. As a workaround, many surgical manipulations such as arterial occlusion or vessel cauterization require constant monitoring of the course of procedure while routinely their progress and eventual success or failure are reported orally or otherwise by an attendant or a specialized device, such as a heartbeat pacer or monitor. Reliance on such an approach is naturally prone to delay in timing of response, potential obscuration by background noises, misinterpretation and other interfering factors of human as well as purely technical nature.

Consequently there is a need for a more timely, precise and reliable delivery of physiological information, especially visual, to a person actively engaged in conductance of procedures or observation under; for example, a surgical microscope. One possible approach to address this problem is by means of an Augmented Reality (AR) approach to presentation of visual and other contextual information. In this modality, relevant pictorial, video and/or any other qualitative or quantitative data are merged with visual, audial or other perceptual field of view of an observer. Such overlay can be done with some delay and, preferably, the minimal one, so that changes in condition and parameters registered are perceived as if occurring in real time and responding to currently observed changes in the sensory field, hence a “Reality”.

This disclosure presents a solution as it pertains to situation in the actual or remotely controlled surgical room. In either scenario, it helps a surgeon or attendant to be more responsive and efficient by preventing disruption of their attention due to turning head and eyes away from a pair of microscope binoculars when observing the surgical field or from a remote display. All of the pertinent visual and textual data, including that confirm validity of projected information can be overlaid within total of visual field observed through imaging system in near-real time or with minimal delay. Thus, a system and method to effectively accomplish such a solution in variety of common imaging modalities employed is disclosed below.

### Embodiments and Applications

The system and method described in this disclosure could be embodied in multiple ways, potentially depending on the application. For example, to assist in open cerebrovascular micro-surgeries, the augmented reality display module may be integrated into a custom-designed eyepiece that replaces a traditional eyepiece of the surgical microscope. Therefore, during procedures such as placing a clip around an aneurysm, the surgeon may benefit from real-time information about blood flow in the field of view presented within the eye-piece itself. In other applications such as open cardiac surgeries that use surgical loupes rather than surgical microscopes, the augmented reality module may be integrated into the surgical loupes such that complementary information is presented overlaid on the view through the surgical loupes. In such a case, the imagery overlaid by the augmented reality module on the field of view is routed through optical waveguides and combined by the image combiner to form the same overlaid imagery on the retina of the viewer. Embodiments of the disclosure may also be embodied such that the surgical microscope is an endoscope, and the augmented reality module overlays additional information on the endoscopic field of view.

### Example Embodiments

An example embodiment of the disclosure includes an augmented reality eyepiece system for a surgical microscope. The system according to the example embodiment includes a processor configured to generate a signal based on image data pertaining to a field of view of the surgical microscope. The system according to the example embodiment includes an eyepiece configured to integrate into the surgical microscope. The eyepiece includes an image generation module configured to generate an image based on the signal received from the processor. The eyepiece includes an image combiner configured to combine the image generated by the image generation module with light received from the field of view. The eyepiece includes visualization optics configured to combine the light received from the field of view and the image generated by the image generation module, and present a combined image to an eye of a user.

In some implementations, the eyepiece according to the example embodiment includes an image splitter configured to split the light received from the field of view into a first portion and a second portion. The image splitter directs the first portion to the visualization optics. The eyepiece according to the example embodiment includes a camera module configured to receive the second portion of the light from the image splitter and generate the image data. The processor is configured to receive the image data and generate the signal with a latency less than or comparable to a persistence of vision.

In some implementations, the system according to the example embodiment includes an illumination source and illumination optics configured to deliver light from the illumination source to the field of view. The eyepiece according to the example embodiment includes an image splitter configured to split the light received from the field of view into a first portion and a second portion, the image splitter directing the first portion to the visualization optics. The eyepiece includes a camera module configured to receive the second portion of the light from the image splitter and generate the image data. The processor is configured to receive the image data and generate the signal with a latency lower than a persistence of vision, and the processor is configured to generate a laser speckle contrast imaging (LSCI) image based on the second portion of light received from the image splitter, and provide the LSCI image to the image generation module via the signal. The image generation module is configured to generate the image based on the signal.

In some implementations, the system according to the example embodiment includes a memory storing information relating to the field of view. The processor is configured to generate the signal according to the stored information, and provide one or more of a textual, numerical, graphical, or image rendering for display by the image generation module. In some implementations, the memory and provision of one or more of a textual, numerical, graphical, or image rendering for display by the image generation module can be implemented in combination with the features of the preceding two paragraphs.

### 1.1 Overview

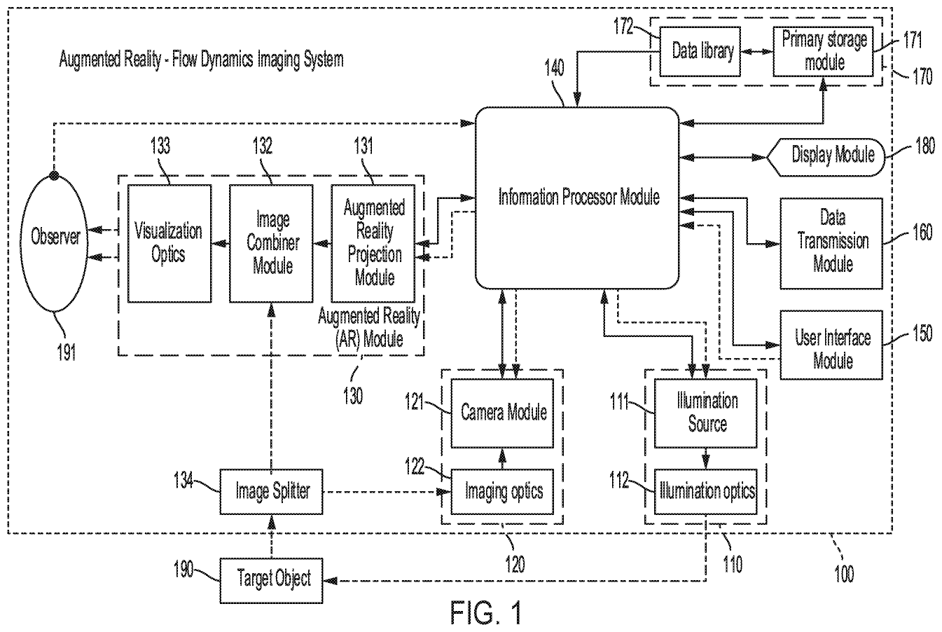

FIG. 1 is a block diagram illustrating an example embodiment of a real-time Augmented Reality (AR) system 100 (enclosed in a short-dashed rectangle) for presentation and examination of underlying physiological information (such as particulate flow, blood-oxygenation map, optical contrast agent imaging, etc.) in a target object 190. In various embodiments, the target object 190 can include any tissue, organ, or organ system of any human or animal biological system, including but not limited to the cornea, sclera, retina, epidermis, dermis, hypodermis, skeletal muscle, smooth muscle, cardiac muscle, brain tissue, the spinal cord, the stomach, large and small intestines, pancreas, liver, gallbladder, kidneys, endocrine tissue, or reproductive organs and associated or disassociated blood vessels and lymph vessels. In various embodiments, the AR system 100 includes at least one Illumination Module (including any associated illumination and beam shaping optics) 110 that is configured to generate at least one type of coherent light and to direct the generated light to the target object 190 being imaged; at least one Image Acquisition (IA) Module 120 that is configured to capture light that is reflected, generated, scattered or re-emitted (by means of fluorescence, phosphorescence, or any other type of Raman scattering or (bio) luminescence) by the target object 190 being imaged; at least one Image Combiner (IC) Module 132 that is configured such that the desired ROI is projected (eventually) on the operator's eye retina by means of Visualization Optics 133 (e.g., eyepiece optics including an ocular lens) within the Augmented Reality Display (ARD) Module 130 with desired specifications of magnification, field of view, speckle size, spot size, frame rate, light flux and optical resolution; at least one Information Processor Module 140 configured at least to estimate anatomical and physiological information in real-time or near-real-time using the data acquired by the IA Module 120 and to control the operation of the whole AR module 130; at least one display module 180 configured to present the estimated anatomical and physiological information or equivalent parameters calculated from the image data by the processor module 140 or the raw data acquired by the IA module 120; at least one data storage module 170 configured to store estimated anatomical and physiological information or equivalent parameters calculated from the acquired images by the information processor module 140 or the raw data acquired by the IA module 120 for temporary or future use; and at least one user interface module 150 configured to allow the user or observer 191 to interact with the AR system 100 and define, pre-set, or program various options, values and settings for features and parameters relevant to the performance of the various modules 110, 120, 130, 140, 160, 170, and 180 of the AR system 100, at least some of which are can be stored, retrieved and saved by means of the Storage Module 170. In some embodiments, the optical signal transmission for acquisition and illumination of the FOV, or ROI of the FOV, to and from the target object 190 comprises of combination of one or more optical elements (lens, filters, beam splitters, etc.), optical waveguides (single-mode or multi-mode optical fibers, rectangular waveguides, etc.), etc.

The Augmented Reality (AR) Module 130 includes an arrangement of one or more light manipulation components, which includes but is not limited to lenses, mirrors, dichroic mirrors, apertures, filters, beam splitters, beam shapers, polarizers, wave retarders, diffractive and adaptive optical elements, fixed and variable phase and/or amplitude masks, analog and/or digital light processors (DLP), microdisplays, visible light sources, micro electro-mechanical system (MEMS) devices and fiber optics, that serve the purpose of delivering optical imaging content from the Augmented Reality Projection (ARP) Module 131 to the Visualization Optics 133, such as e.g. a microscope eyepiece or a Head-Mounted Display (HMD). The various embodiments of AR Module 130 include components that manipulate the light in a manner than is useful for imaging modality of interest based on the specific application. In some embodiments, the optical assembly for Image Combiner module 132 includes polarizers, depolarizers, neutral density filters, waveplate retarders and/or polarizing beam splitters in the imaging paths from ARP Module 131 and Imaging optics 122 that polarize or depolarize the light in a manner that is optimally combined with the light coming back from the target object 190 and directed towards Visualization Optics 133.

### 1.2 Systems and Modules

### 1.2.1 Illumination Module

The illumination module 110 includes two sub-modules 1) illumination source 111 and 2) illumination optics 112.

The illumination source 111 includes one or more light sources such that at least one of the sources produces coherent light (e.g., a laser) for speckle production and LSCI. In some embodiments, the illumination source 111 includes additional light sources that produce coherent, incoherent, or partially coherent light. The wavelength of the one or more lights being emitted by the light sources in an example embodiment lies in the 0.1 nm (X-ray) to 10,000 nm (micro-wave) range. In some embodiments, one or more wide-band light sources is used to produce light with more than one wavelength. In some embodiments, the one or more wide-band light sources is fitted with one or more filters to narrow the band for specific applications. Typically, incoherent illumination sources are useful for reflectance- or absorption-based photography. In some embodiments, direct visualization and focusing of the AR system 100 on the target object 190 is achieved under incoherent illumination. In some embodiments, the illumination source 111 incorporates mechanisms to control one or more of the power, intensity, irradiance, timing, polarization or duration of illumination. Such a control mechanism may be electronic (examples include a timing circuit, an on/off switching circuit, a variable resistance circuit for dimming the intensity, or a capacitor-based circuit to provide a flash of light) or mechanical where one or more optical elements (examples include an aperture, a shutter, a filter, or the source itself) may be moved in, along or out of the path of illumination. In various embodiments, the light sources included in the illumination source 111 may be pulsatile or continuous, and polarized partially, linearly, circularly, or randomly (non-polarized). They can be based on any laser, plasma discharge (flash), luminescence phenomena, incandescent, halogen or metal vapor (e.g. mercury) lamp, light emitting diode (LED or SLED (super-luminescent LED)), X-ray, gamma-ray, Charged Particle (e.g. Electron) Accelerator, Variable Electromagnetic Field (such as those used in magnetic resonance imaging or spectroscopy) or other ionizing or non-ionizing radiation and technology.

The second sub-module of illumination module 110, the illumination optics 112, includes an arrangement of one or more light manipulation components, which includes but is not limited to lenses, mirrors, apertures, filters, beam splitters, beam shapers, polarizers, wave retarders, and fiber optics, that serve the purpose of delivering light from the illumination module 110 to the desired ROI in the target object 190. In some embodiments, additional light manipulation elements such as optical diffraction element can be used to project a pattern onto the target. Optical diffraction element can be configured to project a light pattern on the target object 190. The illumination optics 112 for the various embodiments includes components that manipulate the light in a manner than is useful for imaging the tissue of interest based on the specific application. In some embodiments, the illumination optics 112 include a polarizer in the path of illumination that polarizes the light in a manner that significantly attenuates the light except when reflected or scattered (depending on operator's preference) by the target object 190.

### 1.2.2 Image Acquisition Module

The image acquisition (IA) module 120 consists of two sub-modules 1) camera module 121 and 2) imaging optics 122 designed to undertake desired imaging schemes such as LSCI, ICG (video-) angiography, other kinds of fluorescence microscopy or imaging modalities.

The camera module 121 includes at least one camera sensor or image acquisition device that is capable of transducing incident light to a digital representation (called image data). In some embodiments, camera module 121 includes at-least two such camera sensors or image acquisition device where one would be used to capture live visible FOV, or ROI of the FOV, of target object 190 while the rest of the acquisition devices are specific for capturing data from FOV, or ROI of the FOV, of target tissue illuminated with one or more coherent light sources. The camera module 121 is configured to direct the image data for further processing, display, or storage. In some embodiments, the camera module 121 includes mechanisms that control image acquisition parameters, including exposure time (i.e., time for which the camera sensor pixel integrates photons prior to a readout), pixel sensitivity (i.e., gain of each pixel), binning (i.e., reading multiple pixels as if it was one compound pixel), active area (i.e., when the entire pixel array is not read out but a subset of it), among others. In the various embodiments, the at least one camera sensor used in the camera module 121 is a charge coupled device (CCD), complementary metal oxide semiconductor (CMOS), metal oxide semiconductor (MOS), array of (avalanche or hybrid) photodiodes, photo-tubes, photo- and electron multipliers, light or image intensifiers, position-sensitive devices, thermal imagers, photo-acoustic and ultrasound array detectors, raster- or line-(confocal) scanners, nipkow-disc or confocal spinning-disc devices, streak cameras or another similar technology designed to excite, detect and capture imaging data.

The imaging optics 122 includes an arrangement of one of more light manipulation components that serve the purpose of focusing the ROI of FOV of the target object 190 on to the at least one camera sensor of the camera module 121. In some embodiments, the imaging optics 122 comprise a means to form more than one image of ROI or sub-regions of the ROI of the target object 190. In some embodiments, the more than one image projects onto the one or more camera sensors or on a retina of an observer 191 through an eyepiece. In the various embodiments, the imaging optics 122 determine the imaging magnification, the field of view (FOV), size of the speckle (approximated by the diameter of the Airy disc pattern), and spot size at various locations within the FOV. In some embodiments, the imaging optics 122 includes light manipulation components that, in conjunction with components of the illumination optics 112, reduce the undesired glare resulting from various optical surfaces. In some embodiments, additional light manipulation control using opto-mechanical components for aperture control for manipulation of speckle size of the image data (e.g., manual or electronics iris), adjustment for depth of focus (e.g., focusing lens), switching filter sets (e.g., including but not limited to electronics slider, filters, polarizers, lens), alignment of focused light plane orthogonal to optical path (e.g., 45° mirrors); some or all of which are connected by wired or wireless means and controlled using the user interface module 150 through the information processor module 140.

### 1.2.3 Information Processing Module

The information processor (IP) module 140 includes one or more processing elements configured to calculate, estimate, or determine, in real-time or near-real-time, one or more anatomical, physiological and functional information and/or related parameters derived from the imaging and other sensor data, and generate a data signal based on image data pertaining to a field of view of the surgical microscope for presentation to the user in context of other information available. The IP module 140 includes one or more processing elements designed and configured to implement control functions for the AR system 100, including control of operation and configuration parameters of the acquisition module 120 and its sub-modules 1) camera module 121 (e.g., exposure time, gain, acquisition timing) and 2) Imaging optics 122 (e.g., iris control, focus control, switching filter sets); the illumination module 110 and its sub-modules 1) Illumination source 111 (e.g., irradiation power, timing, duration, and synchrony of illumination) and 2) Illumination optics 112 (e.g., focus control); control of the transmission of image data or derivatives thereof to and from the display module 180, the User interface module 150 (preview of image data) and/or the storage module 170 and a data transmission module 160; control of which anatomical, physiological and functional information and/or related parameters should be calculated, estimated, or determined by the processor module 140; control display or projection of the same by Display Module 180 and AR module 130 and its sub-modules; and control of the overall safety criteria, sensors, interlocks and operational procedures of the AR system 100. In some embodiments, the information processor module includes subroutines for machine learning (supervised (task-driven), unsupervised (data driven) and some cases reinforcement (algorithm react to an environment/event)) which leverages access to information from one or more such as image data and other sub-modules such has 110, 120, 130, 160, and 170.

In various embodiments, the information processor module 140 is configured to calculate, estimate, or determine one or more anatomical and physiological information or equivalent parameters calculated from the image data in one or more of the following modes:

Real-time video mode—In the real-time video mode, the information processor module 140 is configured to calculate, estimate, or determine one or more anatomical and physiological information or equivalent parameters calculated from the image data based on certain predetermined set of parameters and in synchrony or near-synchrony with the image acquisition. In the real-time video mode, the frame rate of the video presented by the display module 160 is greater than 16 frames per second (fps), allowing the surgeon to perceive uninterrupted video (based on the persistence of vision being 1/16^{th} of a second).

Real-time vessel mode—In real-time vessel mode, the AR system 100 is configured to allow the surgeon to select, using automatic or semi-automatic means, one or more vessels and to emphasize the anatomical and physiological information in the selected vessels over other vessels in the field of view (FOV). In some embodiments, the AR system 100 is configured to allow the surgeon to select all arteries or all veins, extracted automatically, in the entire FOV, or a region of interest (ROI), of the FOV. In such embodiments, the extraction may be achieved by either (a) computing the anatomical or physiological information in the entire field but displaying only the anatomical or physiological information in the selected vessels, or (b) computing the anatomical or physiological information only in the selected vessels and displaying the anatomical or physiological information accordingly, or (c) computing the anatomical or physiological information in the entire field and enhancing the display of the selected vessels through an alternate color scheme or by highlighting the pre-selected vessels centerlines or edges.

Real-time relative mode—In the real-time relative mode, the processor module 150 includes the baseline values of anatomical and physiological information in its computation of instantaneous values of anatomical or physiological information. The real-time relative mode may be implemented as a difference of instantaneous values of anatomical or physiological information from the baseline values, or as a ratio of the anatomical or physiological information with respect to baseline values.

Snapshot mode—In the snapshot mode, the processor module 150 generates a single image of the anatomical or physiological information in the surgical FOV. In this embodiment, the processor module 150 may utilize a greater number of frames for computing the anatomical or physiological information than it utilizes during the real-time modes, since the temporal constraints are somewhat relaxed. In the snapshot mode, all the functionalities of the real-time modes are also possible (e.g., display of change of blood flow instead of blood flow, or enhanced display of a set of selected vessels).

### 1.2.4 Display Module

The display module 180 comprises one or more display screens or projectors configured to present the estimated anatomical and physiological information or equivalent parameters calculated from the image data by the information processor module 140; augmented overlaid image data of AR module 130 which contains processed image data projected using AR projection module 131 overlaid onto unobstructed FOV, or ROI of the FOV, (from one arm of image splitter 134) of the target object 190); or the raw data acquired by the acquisition module 120. In some embodiments, the one or more display screens can be physically located in close proximity to the remaining elements of the AR system 100. In some embodiments, the one or more display screens are physically located remotely from the remaining elements of the AR system 100. In the various embodiments, the one or more display screens are connected by wired or wireless means to the processor module 140. In some embodiments, the display module 180 is configured to provide the observer with a visualization of the ROI and the estimated anatomical and physiological information or equivalent parameters calculated from the image data by the information processor module 140. In the various embodiments, the display module 180 is configured for real-time visualization, near-real-time visualization, or retrospective visualization of imaged data or estimated anatomical and physiological information or equivalent parameters calculated from the image data that is stored in the storage module 170. Various aspects of anatomical and physiological information, or equivalent parameters and other outputs of the processor may be presented in the form of monochrome, color, or pseudo-color images, videos, graphs, plots, or alphanumeric values.

### 1.2.5 Storage Module

The storage module 170 includes one or more mechanisms for storing electronic data, including the estimated anatomical and physiological information or equivalent parameters calculated from the image data by the processor module 140, overlaid eye-piece image data from the image combiner module 132 of the AR module 130, or the raw data acquired by the acquisition module 120. In various embodiments, the storage module 170 is configured to store data for temporary use in a primary storage module 171, and for long-term use the data can be transferred to a data library module 172. In various embodiments, the storage module 170 and/or the data library module 172 can include random access memory (RAM) units, flash-based memory units, magnetic disks, optical media, flash disks, memory cards, or external server or system of servers (e.g., a cloud-based system) that may be accessed through wired or wireless means. The storage module 170 can be configured to store data based on a variety of user options, including storing all or part of the estimated anatomical and physiological information or equivalent parameters calculated from the image data by the processor module 140, the raw data acquired by the acquisition module 120 or the system information like setting or parameters used while recording/capturing the raw or processed image data from the user interface module 150.

The storage module 170 includes two sub-modules: 1) the Primary storage module 171 and 2) the Data library module 172. The primary storage module 171 can embody of all the functionality discussed for the storage module 170 above when the image data and the system data information are captured/stored during the working of the AR system 100 for temporary use. The data and/or system information and parameters that are useful for future AR system 100 operations (e.g., adjustments of system parameters, optical or mechanical corrections) can be transferred to and stored in the data library module 172. In various embodiments, the transferring of data can be done using one or more mechanisms which includes random access memory (RAM) units, flash-based memory units, magnetic disks, optical media, flash disks, memory cards, or external server or system of servers (e.g., a cloud-based system) that may be accessed through wired or wireless means.

### 1.2.6 User Interface Module

The user interface module 150 includes one or more user input mechanisms to permit the user to control the operation and settings of the various modules 110, 120, 130, 140, 160, 170, 180 and their sub-modules of the system 100. In various embodiments, the one or more user input module includes a touch-screen, keyboard, mouse or an equivalent navigation and selection device, and virtual or electronic switches controlled by hand, foot, one or both eyes, mouth, head or voice. In some embodiments, the one or more user input mechanisms is the same as the one or more display screens of the display module 180.

### 1.2.7 Augmented Reality (AR) Module

The Augmented Reality (AR) module 130 includes three sub-modules: 1) an Augmented Reality Projection (ARP) module 131, 2) an Image Combiner (IC) module 132, and 3) Visualization Optics 133.

The Augmented Reality Projection (ARP) module 131 includes one or more miniaturized projection display or screens configured to present the estimated anatomical and physiological information or equivalent parameters calculated from the image data by the information processor module 140 and provided to the ARP module 131 in the form of a data signal. The data signal can include data pertaining to one or more of image sequences, graphical representations, numerical values, or text-based representations. In some embodiments, miniaturized projection display (ARP-display unit) includes of one of many micro-displays made from liquid crystal display (LCD) or its derivatives, organic light-emitting diode (OLED) or its derivatives or digital light processing (DLP). In some embodiments, the processed image data is communicated/transferred to the ARP-display unit of the ARP module using optical elements (e.g., optical fiber bundle) or electronics (e.g., wire or wireless). Various aspects of anatomical and physiological information, or equivalent parameters and other outputs of the processor may be presented in the form of monochrome, color, or pseudo-color images, videos, graphs, plots, or alphanumeric values onto the ARP-display unit of the ARP module. In some embodiments, the ARP module 131 incorporates mechanisms to control one or more of the brightness, alignments, timing, frame rate, or duration of image data. Such a control mechanism may be electronic (examples include a timing circuit, an on/off switching circuit, a variable resistance circuit for dimming the brightness, dedicated microcontroller/microprocessor (evaluation boards) or a capacitor-based circuit) or mechanical where one or more optical elements (examples include an aperture, a shutter, a filter, or ARP-display unit itself) may be moved in or out of the path of projection onto the Image combiner (IC) module 132.

The IC module 132 includes of arrangement of one or more light manipulation components or relay optics, which includes but is not limited to lenses, mirrors, apertures, filters, beam splitters, beam shapers, polarizers, wave retarders, neutral density filters and fiber optics, that serve the purpose of refining and delivering light containing the processed image data from the ARP-display unit of the ARP module 131 onto the visualization optics 133 (e.g., eyepiece optics) to be observed in real-time or near real-time by the observer (e.g., surgeon, technician). Some embodiments may include arrangement of one or more opto-mechanical components such as the electronic or manual iris (to control system aperture), lenses, 3D or 2D optical translation stages, optical rotary stages, etc. The purpose of opto-mechanical components is finely tune/adjust the magnification, alignment along rotational, orthogonal and depth plane with respect to the optical light coming from the ARP module 131. The purpose of the IC module 132 is to combine (or overlay) the processed image data onto the unobstructed FOV, or ROI of the FOV, of the target object 190, thus creating a combined image. Thus, the estimated anatomical and physiological information or equivalent parameters calculated from the image data is presented over and along with the unobstructed FOV, or ROI of the FOV, of the target object 190 to the observer through the visualization optics 133. In some embodiments, additional arrangement of above mentioned optical and opto-mechanical components can be used to relay the overlaid (combined) image data to the eyepiece camera.

Visualization optics 133 includes of arrangement of one or more light manipulation components or relay optics, which includes but is not limited to lenses, mirrors, apertures, filters, beam splitters, beam shapers, polarizers, wave retarders, neutral density filters and fiber optics, that serve the purpose of delivering combined image from the image combiner (IC) module 132 to relay the information to observer's retina (e.g., surgeon, technician). The purpose of the visualization optics 133 is to match the magnification, depth of perception of the FOV, or ROI of the FOV, of the combined image data relayed from the IC module 132.

### 1.2.8 Data Transmission Module

The data transmission module 160 includes one or more input/output data transmission mechanisms (wired or wireless) to permit the user to transmit and receive information between the system 100 and a remote location or other system. The information includes systems parameters, raw and/or processed image data, etc.

### 1.2.9 Image Splitter

The image splitter (IS) module 134 can include imaging optics leveraged from the optical assembly of a surgical microscope (for example, Zeiss OPMI series, Leica Microsystems M- and MS-series, and similar microscopes) or a physically-integrated surgical microscope. In some embodiments, one or both optical paths within the surgical microscope can be integrated with the augmented reality (AR) module 130, thus achieving mono- or stereo-AR eye-piece capabilities. This integrated system would have the ability to estimate particulate flow within a FOV, the size of which is determined by the magnification settings of the surgical microscope. The system 100 can estimate the particulate flow within the depth of focus as set by the surgical microscope. When used in human surgical environments, the FOV has a diameter that ranges from approximately 10 mm to 50 mm in diameter. When used in veterinary environments, the FOV has a diameter that ranges from approximately 5 mm to 50 mm in diameter.

### 1.3 Augmented Reality Eyepiece Display

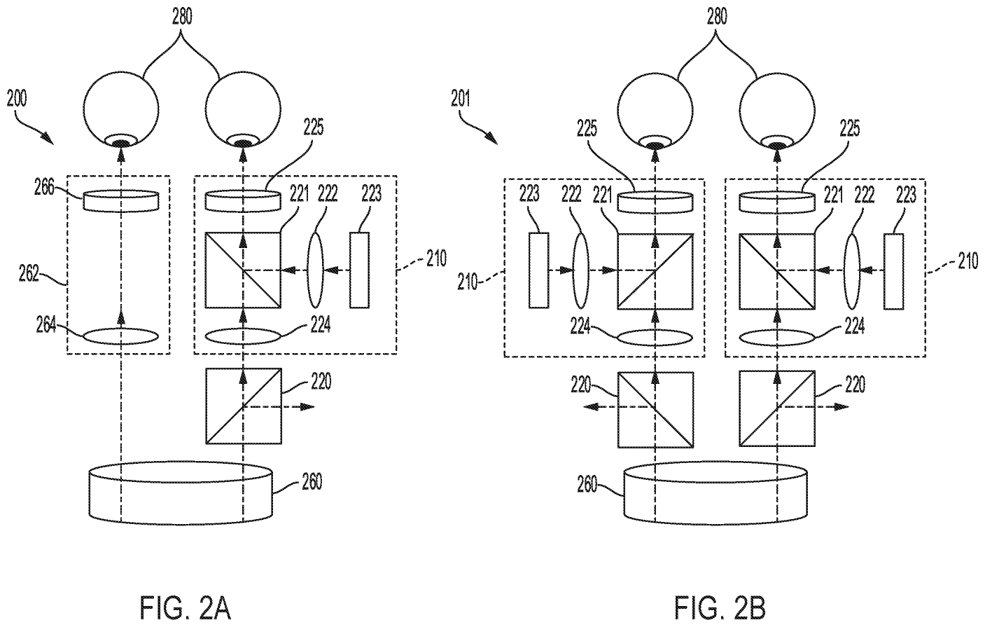

FIGS. 2A and 2B depict two possible implementations 200 and 201 of the Augmented Reality (AR) module 210 in a surgical microscope 260. The AR module 210 can be, for example, the AR module 130 previously described. The AR module 210 (including a beam splitting element 220) can replace either or both of the surgical microscope's 260 stock eyepieces 262, including the stock eyepiece's visualization optics 266 and objective lens optics 264. While the FIG. 2A implementation 200 achieves mono-AR eye-piece capabilities, the FIG. 2B implementation 201 can provide stereoscopic projection of AR information to an observer 280 of the stereo surgical microscope 260. In some implementations, the AR module 210 includes the beam splitting element 220, which can receive light from a field of view of the surgical microscope 260, and divert a portion of the received light to a an image acquisition module of the AR system; for example, the camera module 121.

The implementation depicted presents AR modules 210 comprised of three sub-modules 1) an Augmented Reality Projection (ARP) module, 2) an Image combiner module, and 3) Visualization Optics (VO) module which are all integrated into one subsystem with a purpose to replace the regular eye-piece 262 of a surgical microscope 260, and have minimal alteration of a light path upstream of that.

The ARP module includes a miniaturized projection display or screen 223 configured to present the estimated anatomical and physiological information or equivalent parameters calculated from the image data by the information processor module and combined with the visual field by means of a beam splitting element 221, to be projected into observer's 280 single eye or both eyes using optical elements such as projection lenses 222.

The Image Combiner (IC) module, represented by the beam splitting element 221 includes an arrangement of one or more light manipulation components or relay optics, which includes but is not limited to lenses, mirrors, apertures, filters, beam splitters, beam shapers, polarizers, wave retarders, neutral density filters and fiber optics, that serve the purpose of delivering light containing the processed image data from the ARP-display unit or units onto the visualization optics 225 (e.g., eyepiece optics including an ocular lens) to be observed in real-time or near real-time by the observer (e.g., surgeon, technician). The beam splitting element 221 can combine the processed image data with light from a field of view of the surgical microscope 260 received via objective lens optics 224 of the AR module 210. In some embodiments, arrangement of one or more opto-mechanical components such as the electronic or manual iris (e.g., control aperture), lens, 3D or 2D optical translation stages, optical rotary stages, etc. The purpose of opto-mechanical components is finely tune/adjust the magnification, alignment along rotational, orthogonal and depth plane with respect to the optical light coming from the projection display 223.

The Visualization Optics (VO) module, represented here by visualization optics 225, includes an arrangement of one or more light manipulation components or relay optics, which includes but is not limited to lenses, mirrors, apertures, filters, beam splitters, beam shapers, polarizers, wave retarders, neutral density filters and fiber optics, that serve the purpose of delivering combined image from the beam splitting elements 221 to relay the information to observer's 280 retina (e.g., surgeon, technician). The purpose of the VO module is to match the magnification, depth of perception of the FOV, or ROI of the FOV, of the combined image data relayed from the IC module or modules mentioned above.

### 1.4 Example of Integrated System Embodiment

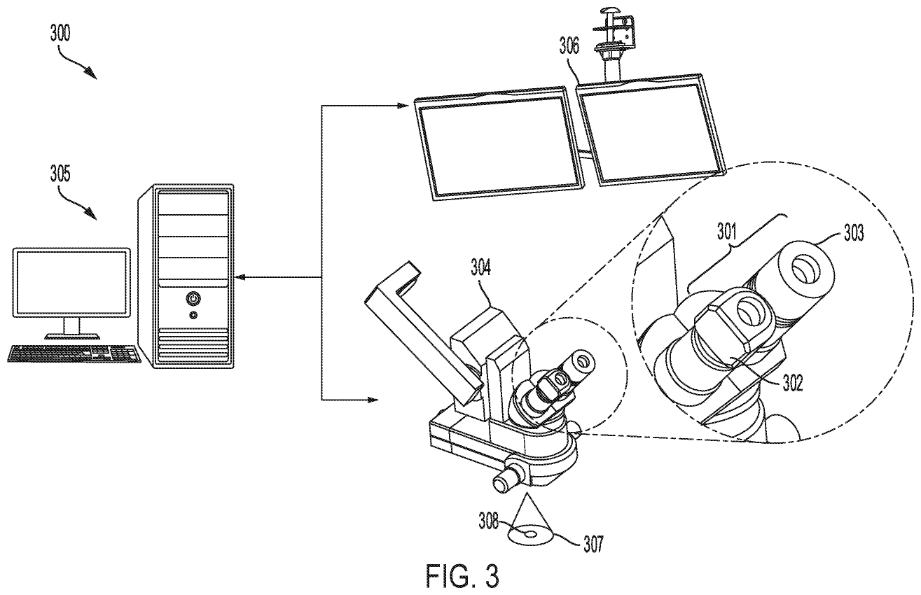

The embodiment in FIG. 3 shows an example system 300 that includes a physically-integrated surgical microscope 304. The illumination optics and imaging optics leverage the optical assembly of the surgical microscope 304. The system 300 estimates one or more anatomical and physiological information or equivalent parameters for blood in form of different quantification index (e.g., flow, velocity, blood hemoglobin oxygenation levels, blood flow-velocity index etc.) within an FOV 307 or ROI 308 of the FOV 307, the size of which is determined by the magnification settings of the surgical microscope 304. In some embodiments, one or more fiber-optic illumination ports may be employed to transmit light to the surgical area to illuminate the ROI 308. When used in human surgical environments, the FOV 307 has a diameter that ranges from approximately 10 mm to 50 mm in diameter. When used in veterinary environments, the FOV 307 has a diameter that ranges from approximately 5 mm to 50 mm in diameter. The surgical microscope 304 utilizes multiple optical ports to engage 1) the imaging optics to form an image of the FOV 307 on the camera sensor of the camera module, and 2) the augmented-reality projection (ARP) module 302 to project the anatomical and physiological information in one or more of the eyepieces 301 of the surgical microscope 304 while presenting FOV, or ROI of the FOV, to naked eye through the eyepiece without the AR-display 303. In some embodiments, an aperture is included in the imaging optics that determines the diameter of the Airy disc (i.e., speckle size) for a given optical system based on its Numerical Aperture and the wavelength of the laser used. The system 300 can employ an illumination module with laser diode of light in the invisible range (700 nm to 1300 nm) to prevent disruption of the surgical field, a uniform beam shaper to achieve uniform top-hat or flat-top illumination that transforms a Gaussian beam of the laser diode into a uniform intensity distribution, and a near-infrared (NIR) polarizer to generate a linearly polarized illumination. In some embodiments, laser diode homogenization and reshaping may be assisted by two orthogonal Powell lenses. In some embodiments, one or more fiber-optic illumination ports may be employed to transmit light to the surgical area to illuminate the ROI 308. The system 300 may include a tablet, or a laptop or desktop computer 305 that can house a processor module configured to estimate anatomical and physiological information in real-time or near-real-time using the data acquired by the camera module and to control the operation of the imaging device; an ARP module 302 configured to present the estimated anatomical and physiological information or equivalent parameters calculated from the image data by the computer 305 or the raw data acquired by the camera module of the imaging device; a storage module, an internal or external sub-module of the computer 305, configured to store the estimated anatomical and physiological information or equivalent parameters calculated from the image data by the processor module or the raw data acquired by the camera module for future use; and a user interface module, a sub-module of the computer 305 or connected to the computer 305, configured to allow the user or operator to interact with various options for features and parameters relevant to the performance of the various modules of the system 300. In some embodiments, the system 300 includes a transmission module that facilitates transmission of electronic data to a remote server or server system for further storage, processing, or display. In some embodiments, the system 300 is designed specifically for imaging of surface or subcutaneous vasculature. In some embodiments, the system 300 is designed specifically for imaging of the vasculature of surgically exposed tissue. In some embodiments, specific parts (e.g., optical elements) of the system 300 may be exchanged with other parts to optimize the system 300 for imaging the vasculature of specific tissue. In some embodiments, the estimated anatomical and physiological information or equivalent parameters calculated from the image data by the computer 305 or the raw data acquired by the camera module can be presented to additional display devices 306 either by using various wired (for example coaxial cable, FireWire, USB2 or USB3, Ethernet etc.) or wireless (e.g. Bluetooth, 802.11 Wi-Fi, 3G, 4G, 5G, LTE, Infrared, etc.) communication technologies.

### 2 Methods

### 2.1 Multi-Modality Combination Imaging and Augmented Reality Information Presentation

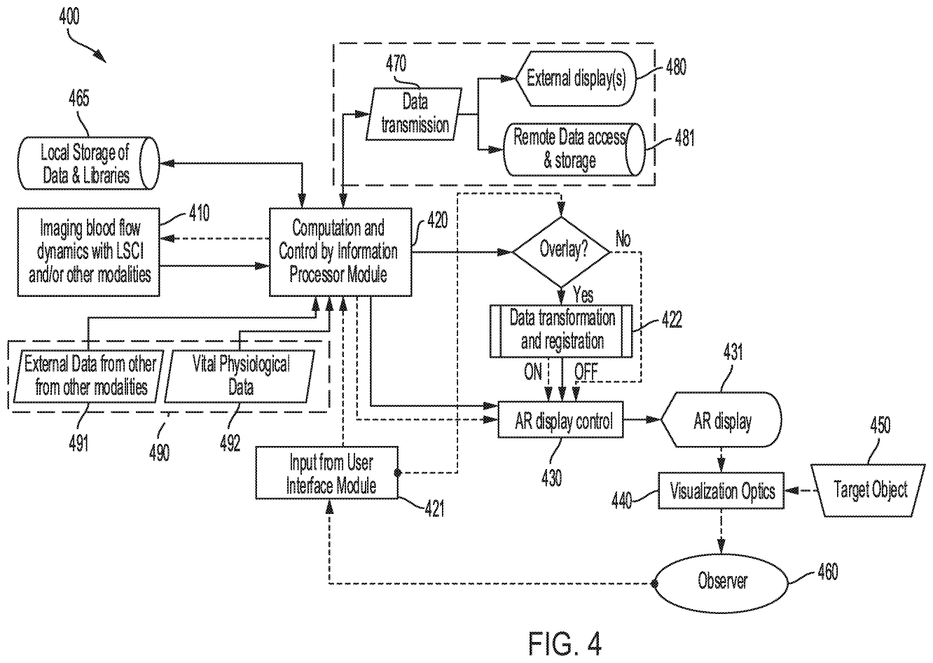

FIG. 4 is a block diagram illustrating an example embodiment of process 400 of operation of a real-time Augmented Reality (AR) system, such as the system shown in FIG. 1. In various embodiments, the method of system execution comprises of at least one imaging modality to obtain anatomical or physiological information (such as blood characteristics, etc.) from the ROI of the target object 450. For example, in order to obtain underlying blood physical or bio-mechanical characteristics, the imaging modality module 410 comprises of one or more of the medical imaging modalities such as Laser-speckle contrast imaging (LSCI), Fluorescence imaging (FL), multi-spectral imaging, and other modalities (such as bio luminance, etc.). In various embodiments, one or more of the imaging modalities can be combined in order to get complimentary information from different imaging modules, depending upon the combination of modalities chosen the setup (including one or more of combination of optical elements, opto-mechanical elements, sensors, etc.) of the acquisition module and illumination module changes. In some embodiments, imaging modalities provides a specific and/or compound information in form of either electro-magnetic (scattering, fluorescence, X-rays, IR etc.) or sound (microwave, ultrasound, etc.) signal or both. This information is recorded and later processed, using Information processor module 420 to obtain various characteristics (estimated anatomical and physiological information or equivalent parameters calculated from the image data) of target object 450. For example, in case of blood circulation within the FOV, or ROI of the FOV, of target, the processed information gives physical and bio-mechanical parameters such as flow, velocity, temperature, blood volume, size of vessels, vasculature, etc. Using the information processor module 420, one or more imaging modalities can the switched sequential or in parallel to trigger/enable one or more of the different Image-modality modules (blood flow imaging, optical contrast imaging, etc.). This enable the user to obtain complimentary information from one or more of the imaging modalities simultaneously to get a comprehensive understating of the underlying physiological processes. The information processor module 420 also has the option for the user to access information from the data library 465 (which includes data and/or system information from previous recording from various direct access storages, sequential access storage, etc.) and from additional data sources 490, such as external data from other modalities 491 (e.g., MRI, CT scan, X-ray, Fluoroscopy, etc.) and vital physiological data 492 (e.g., body temperature, ECG, EEG, SPO2, etc.). In various embodiments, the information processor module 420 can access the data library 465 using communication types either wire (optical fiber cables, BNC cables, Firewire, USB2, USB3, Ethernet cables etc.) or wireless (802.11 Wi-Fi, Bluetooth, 3G, 4G, 5G, LTE, etc.) or both. It can also have a sub-module that can communicate with medical devices (pulse oximeter, bed-side monitor, ECG/EEG machine, etc.) to get direct access for information (such as body temperature, heart rate, blood SPO2, etc.). Once the information is collected from different modalities, data library and other medical devices, the information processor module checks 421 for a user input from the observer 460 (and/or other personal such as attendant) to switch the AR display 431 ‘ON’ or ‘OFF’. This allows the user to freely obtain the information on demand in the visualization optics 440. In various embodiments, the observer can control the switching of the AR display 431, via AR display control 430, the user interface module which is connected to Information processor module 420. If the user chooses 421 to switch ‘ON’ the AR display, the information processor module 420 process the collected information and applies a predetermined subroutine to convert the data into acceptable form (such as text, graph, image, color, etc.) using the data transformation and registration module 422. In various embodiments, the data transformation and registration module consist of analysis routines (mean, standard deviations, etc.), transformation routines (co-ordination transformation, pseudo-color maps, color/background/illumination matching, etc.) and registration routines (co-registration algorithms, etc.). In various embodiments, data can be presented in the eye-piece or the visualization optics 440 from different modalities in different formats overlaid onto the unobstructed FOV, or ROI of the FOV, of the target object 450. On the other hand, the observer 460 (such as a user, surgeon, attendant, etc.) can invoke AR display 431 to switch ‘OFF’ which will not present the observer with the information in the visualization optics 440 and still get an unobstructed view of the FOV, or ROI of the FOV, of the target object 450. In some embodiments, the process 400 includes transmitting and/or receiving 470 data or information from remote locations. The data transmitted to remote location can be displayed by an external display 480 and can also be stored in remote data access and storage (direct access storage, sequential access storage, etc.) 481.

### 2.2 Method for Laser-Speckle Contrast Imaging

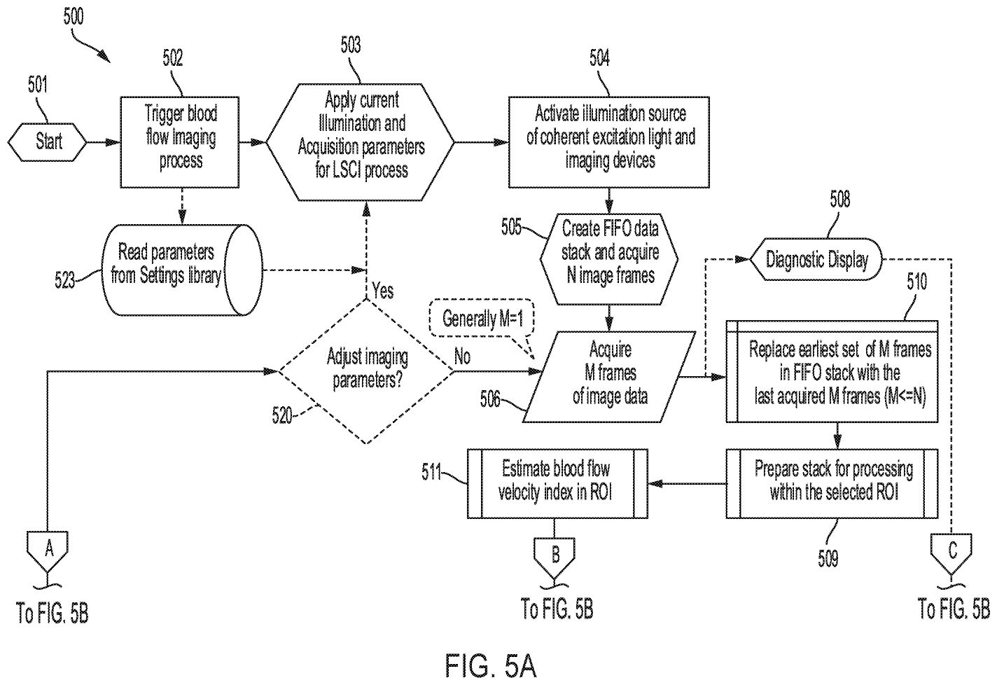

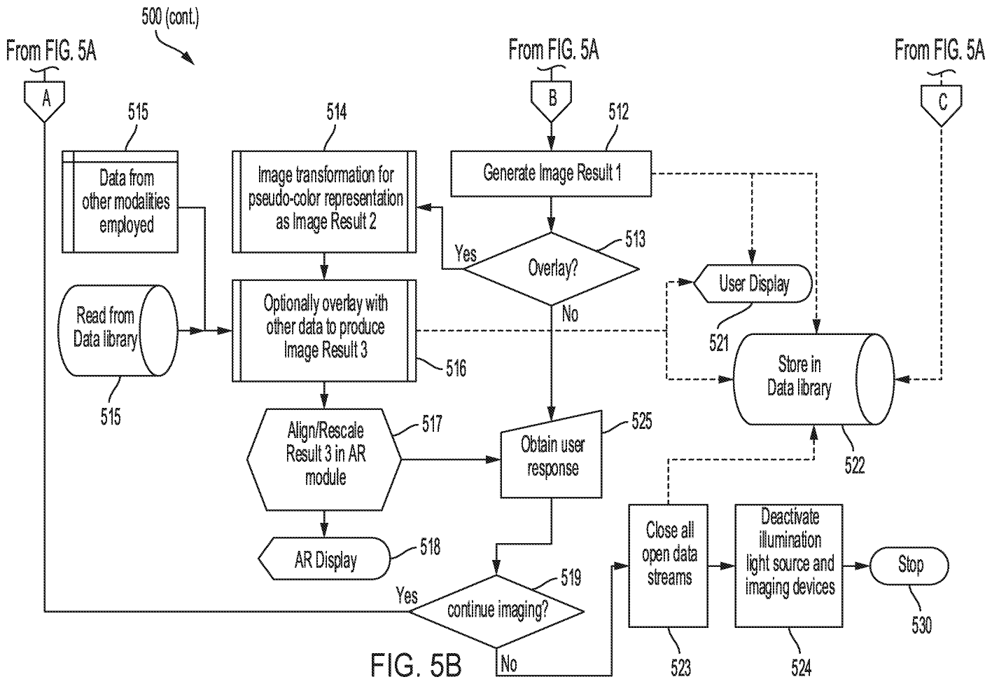

FIGS. 5A and 5B show a flowchart depicting an example embodiment of a process 500 for rapid examination of particulate flow using Laser speckle contrast imaging (LSCI). In this embodiment, the LSCI process for real-time examination of red blood cell flow (blood flow) in live tissue or organism is waiting to start 501 and begins once triggered 502 by an associated event. In various embodiments, the trigger 502 that starts the LSCI process can be manual (i.e., user-generated), automated (i.e., system-generated), or semi-automated (i.e., user- or system-generated). Once the triggered, the subroutine implementing the LSCI method obtains 507 from a stored library 522 the necessary system parameters for acquisition and illumination devices including but not limited to illumination power, duty cycle, sensor exposure time, frame rate, resolution, binning factor, digitizer gain, optical filter selection, etc. Specific to the LSCI method, in one embodiment, a diameter of a variable iris situated in an objective back aperture conjugate plane is harmonized with current magnification setting of the optical system such that scattered laser speckle pattern size matches the camera pixel size used for its detection. The various parameters can be provided by either the user or obtained from presets in computer memory or data library 522. Parameters may be modified manually or automatically using feedback from the imaging result and quality of one or more electronic data registered in real-time during image acquisition. The system then applies these parameters to hardware at 503 and then, at 504, activates illumination of the ROI of the target object with coherent source of light. Next, at 505, the information processor module collects first N initial frames of the stack with the pre-determined exposure time and gain, and then employs rolling a first in-first out (FIFO) acquisition algorithm, acquiring at 506 the specified M number of frames, which is not greater than depth of stack N. Then at 510, the top M (older) frames of the stack are eliminated from the buffer, while newly acquired M frames are added to the bottom. Raw data from the last M frames are also optionally saved to the library 522 and optionally displayed in a diagnostic display 508. Next, using the newly arrived data in the stack, information processing module optionally performs additional image processing steps 509 on the newly acquired set of M frames, potentially including but not limited to background and offset subtraction, bad pixel removal, outlier rejection, denoising, bandpass filtering, smoothing and artifact detection and elimination (masking), followed by optional registration, alignment, and averaging steps in space and/or time domains, to produce a map of scattered laser light intensities. Then, for each pixel belonging to the previously defined ROI, the system employs a subroutine 511 to calculate values for laser speckle contrast K (either in space or time domain or both) and then, if chosen estimates particulate velocity, or flow rate, or any other quantity (index) which may be a linear or non-linear function of K, such as blood flow velocity index (BFVI). Next, through conversion into an appropriate integer or real number space with suitable bit depth, a monochrome LSCI image is generated in the following step 512 as an Image Result 1 and optionally projected to user displays at step 521. Next, information processor checks for user preference at 513 whether to project the Image Result 1 by means of the AR display 518. If ‘Yes’, at 514, the system converts Image Result 1 to its pseudo-color representation (Image Result 2), according to a user-specified manually or computer-generated color and brightness table (palette) providing for intuitive and effective presentation of Image Result 1 as a color picture to be overlaid on top of observer's visual field. Next at 516 this Image Result 2 is combined with any other visual and textual information specified by the user to generate a compound image (optionally including data from other modalities or vitals monitoring data, etc.—optionally read from data library—at 515), and may include additional processing steps including but not limited to background and offset subtraction, outlier rejection, denoising, bandpass filtering, smoothing, artifact elimination as well as registration and alignment. The resulting Image Result 3 is directed to AR module 517, and depending on the user-selected or preset setting, to storage library 522 and user display at 521. Next the AR module 517 converts the digital Image Result 3 to properly scaled and rotated optical signal, and provides the LSCI data in the data signal sent from the processor for projection within AR Display 518. By utilizing parallel computing techniques (including but not limited to FPGA, GPU, analog processors, Machine Vision, Deep Learning) as well as methods based on estimation rather than exact computation of quantities of interest, LSCI calculations and post-processing of the data stack can be finished before another set of frames is integrated from the imaging sensor, and transferred to a memory. Based on the parameter settings at 503 and the user selection at 513, the LSCI process may skip directly to obtaining a user response 525, and decide whether to start another cycle. Next, at 519, if ‘Yes’ is selected, i.e., to continue imaging, (which, in some implementations, can be selected by default), the process continues with a new image acquisition and processing cycle providing real-time examination of particulate flow. It may however, optionally adjust imaging parameters, based on pre-programmed criteria or when settings changed manually by user at step 520. Alternatively, when ‘No’ is selected at 519, i.e., to discontinue imaging, the system goes on to optionally save all data accumulated in data library 522, close all data streams 523 and deactivate imaging devices along with coherent illumination source 524. In parallel to this process, all the data arriving from step 506, 512, 516, and 523 are optionally saved to computer random-access memory and then saved in file or streamed to permanent storage solution or a remote destination for archival and potentially more detailed processing and analysis at 522. The process thus concludes at step 530.

### 2.3 Method for Imaging Based on Other Modalities

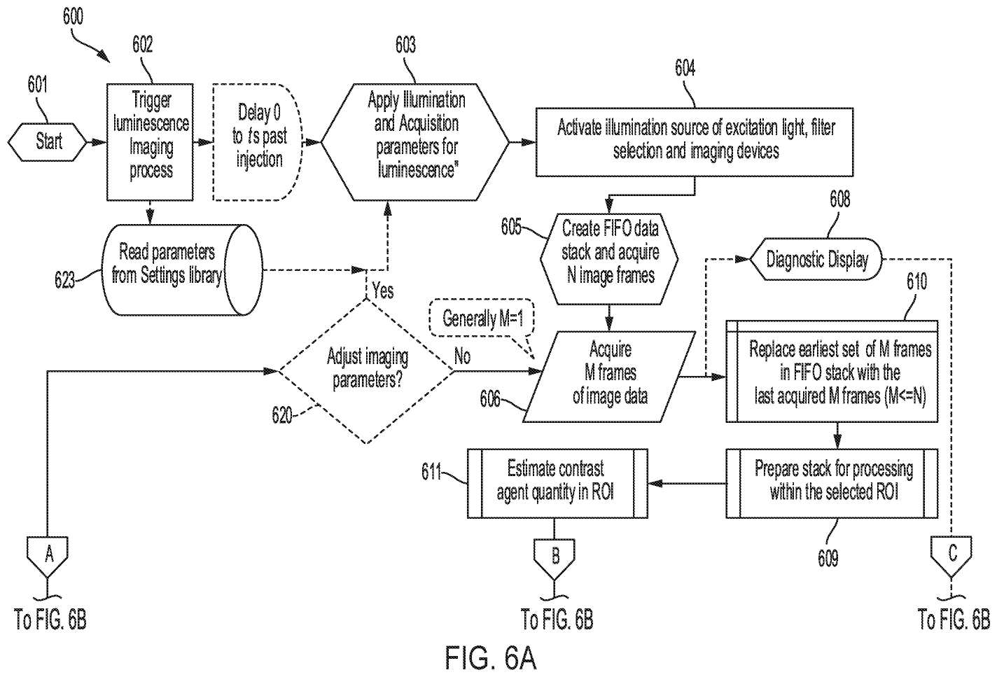

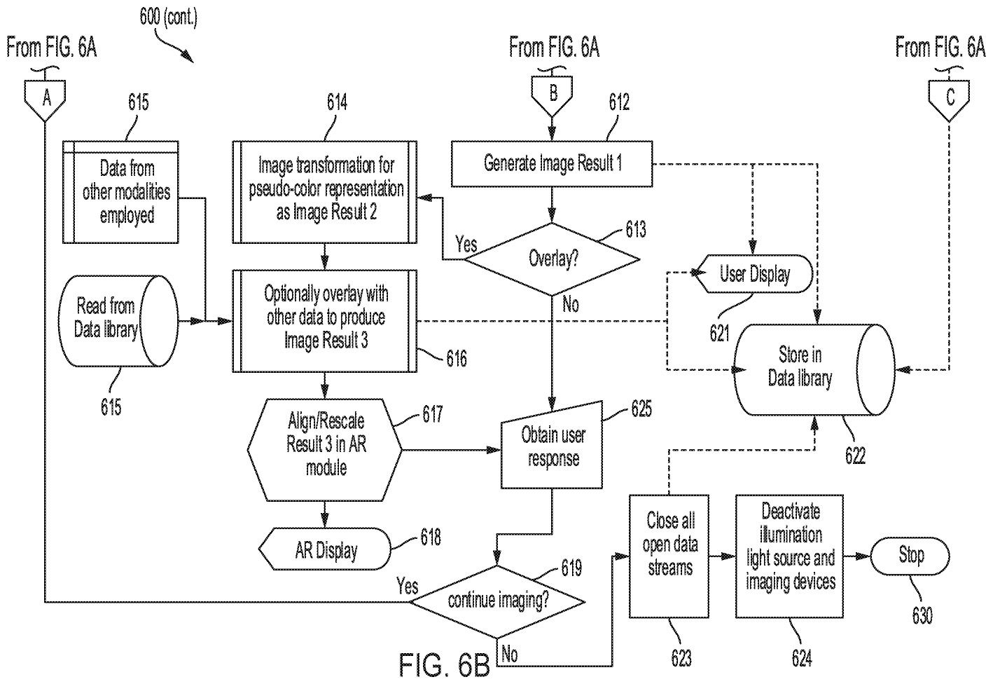

FIGS. 6A and 6B show a flowchart depicting an example embodiment of a process 600 for rapid examination of a target object employing other imaging modalities. The other imaging modalities can include one or more of: spontaneously or externally triggered (uncaged) or excited (bio) luminescence, second or third harmonic generation, phosphorescence, fluorescence (including pO₂, pH, NADH, NAD⁺, FAD⁺, ATP and other vital molecule imaging using fluorescence and phosphorescence probes). In some implementations, the imaging modalities can include any other type of light or sound wave scattering or re-emission phenomenon, such as any type of Raman (classic, resonant, coherent, stimulated, etc.), Raleigh (polarized, coherent (OCT), acousto-optical, opto-acoustic, echo ultrasound, X-ray diffraction or phase-contrast etc.), or scattering (one- or multi-photon excited fluorescence in one of the example embodiments involving use of such agents as solutions of Fluorescein- or Indocyanine Green (ICG)-based dyes). Especially ICG-based (other names used: IC-Green, Cardiogreen, Fox green) near-infrared (NIR) fluorescence embodiment proved to be useful due to following factors: 1) ICG dye has been extensively tested in vivo and is already approved by FDA for human use; 2) its NIR excitation wavelength is away from absorption maxima of most tissue constituents, such as blood hemoglobin and muscle myoglobin; 3) longer (compared to ultra-violet and visible light) excitation and emission wavelengths are characterized by much lower scattering cross-section, thus deeper penetration into biological tissue; 4) ICG fluorescence in the NIR region of spectrum permits simultaneous use of visible light in the operating room and thus simultaneous color videography without any significant interference with process of angiography; and 5) availability of various types of sufficiently bright NIR lasers operating at wavelengths within ICG absorbance band permits simultaneous application of LSCI method (see preceding section above). The process 600 begins at the start 601. In this embodiment, first, if and when required, a relatively small bolus of concentrated dye solution is injected into the flow system. Then, a fluorescence excitation and detection process for rapid examination of amount of a fluorophore present (either of exogenous or endogenous or both) is triggered 602. In various embodiments, the trigger 602 that starts the fluorescence excitation and detection process 600 can be manual (i.e., user-generated), automated (i.e., system-generated), or semi-automated (i.e., user- or system-generated). Once triggered 602, the system that implements the fluorescence imaging (such as ICG video-angiography) process 600 proceeds to obtain 607 and apply 603 the parameters at, including but not limited to illumination power, duty cycle, time delay, sensor exposure time, frame rate, resolution, binning factor, and digitizer gain. In contrast to the LSCI method, a fluorophore-specific fluorescence emission-passing filter can be set in front of the camera sensor to reject any other kind of light and reduce any background. Also, an aperture, if present, can be maximized or taken out of the optical path to preserve the relatively weak fluorescence signal. Some imaging devices also may apply a dedicated NIR imaging mode, or activation of their intensifier or background suppression subsystem. The various parameters can be provided by either the user or obtained from presets in computer memory. Parameters may be modified manually or automatically using feedback from the imaging result and quality of one or more electronic data registered in real-time during image acquisition. To note, just before 603 or 604, a delay can be introduced to the system to keep the system in standby for one or more manual processes such as injection or introduction of an optical contrast agent (such as a fluorescence agent) into the blood circulation to complete. The system then applies these parameters to hardware. At 604, the system activates illumination of the ROI of the target object with appropriately selected and/or filtered source of light to be absorbed by fluorescence contrast agent of choice; for example, an ICG dye.

Once the image frames are acquired by the acquisition module, the information processor module employs a rolling FIFO acquisition algorithm, at 605, then acquires the specified number of frames Mat 606. The next step is to check whether the number of M frames is equal to a predetermined number N frames. If the M is less than N, the system waits for the collection or acquisition of M frames at 606 followed by generation of N frame stack at 610 and preparation of the stock for processing within the selected region of interest at 609. In either case, an N frame stack is generated at step 610. Raw data from the last M frames are also optionally saved to the library 622 and optionally displayed in a diagnostic display 608. Next, this loop restarts and, while awaiting for a next M frames to arrive, the system employs a subroutine, at 611-618, to apply one or more of the Image processing algorithms such as image enhancement, registration, segmentation, etc., for the pixels of interest in the field of view, using the newly arrived data in the stack of M frames of acquired fluorescence intensity data in the buffer, estimating contrast agent quantity within the region of interest at 611, generating a monochromatic brightness Image Result 1 at 612, and also potentially, if overlay is desired 613, computing particulate velocity, perfusion rate or flow or any other quantity (index), which may be a linear or non-linear function of them. At 614, the system converts Image Result 1 to its pseudo-color representation Image Result 2, according to a user-specified manually or computer-generated color and brightness table (palette), providing for intuitive and effective visualization of perfusion, flow information, or actively perfused vasculature and related characteristics (angiogenesis, hemorrhaging, occluded vessels, etc.), and potentially overlays it with other imaging modalities, which may necessitate additional processing steps, including but not limited to background and offset subtraction, outlier rejection, denoising, bandpass filtering, smoothing, and artifact elimination. Process step 616 optionally implements a subroutine to convert and transform generated image result 614 and other data (from data library 615—comprising of data from other modalities or vital monitoring data, etc.) to compound image data with different data represented in different format thereby presenting more than one image modality data in Image Result 3. The system forwards Image Result 2 or Image Result 3, with aligning/rescaling 617 as appropriate, depending on the user-selected or preset display setting to AR module 618. In some implementations, the system can utilize techniques to accelerate processing, including parallel computing, analog processing, Machine Vision, Deep Learning, and processing techniques based on estimation rather than exact computation of quantities of interest calculations. In such implementations, post-processing for the stack can be finished before another set of frames is integrated on the imaging device and transferred to computer memory at 606. Based on the parameter settings at 607, the fluorescence excitation and detection process 600 continues with image acquisition and processing cycles providing real-time examination of target object. In parallel to this process, all the raw data arriving from steps 606, 612, 616, and 623 can be saved to computer random-access memory, or streamed to permanent storage solution or a remote destination for archival and potentially, at 622, more detailed processing and analysis. Based on the parameter settings at 607 or the user input at 625, the imaging process checks to enable or disable, at 617, AR module 618. Next, at 619, if ‘YES’, the process 600 continues with image acquisition and processing cycle providing real-time examination of chosen ROI, checking whether current imaging parameters should be adjusted at 620. If ‘NO’, the process 600 terminates at 630. During termination of imaging process, 624 switches off the illumination source. In some embodiments, either or both of the Image Result 2 or 3 can be sent to be displayed in external user display devices (TV, monitors, 3D-OLED, etc.) at 621.

### 2.4 Method for Multi-Spectral Imaging

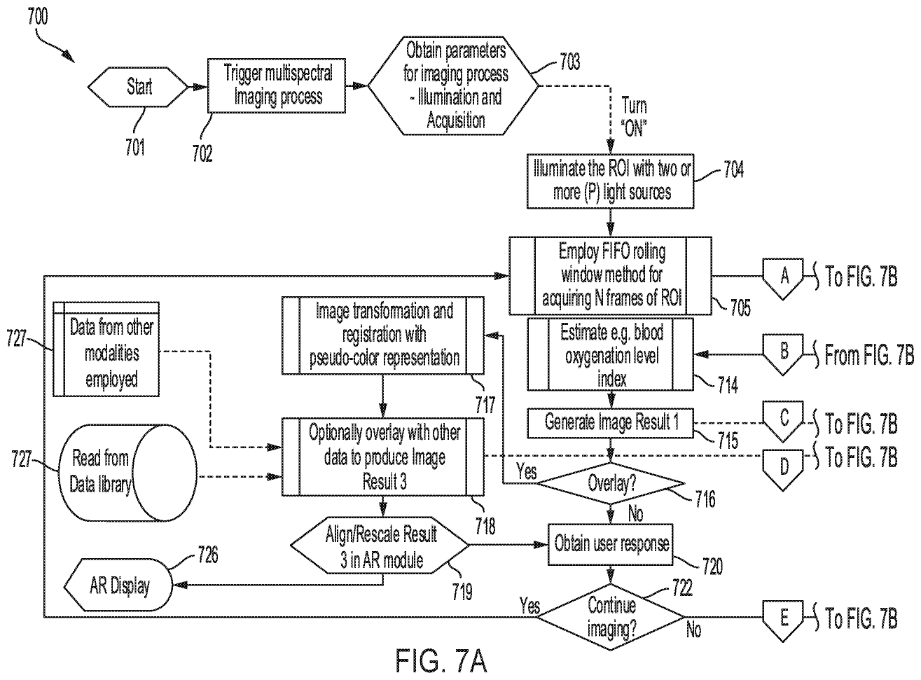

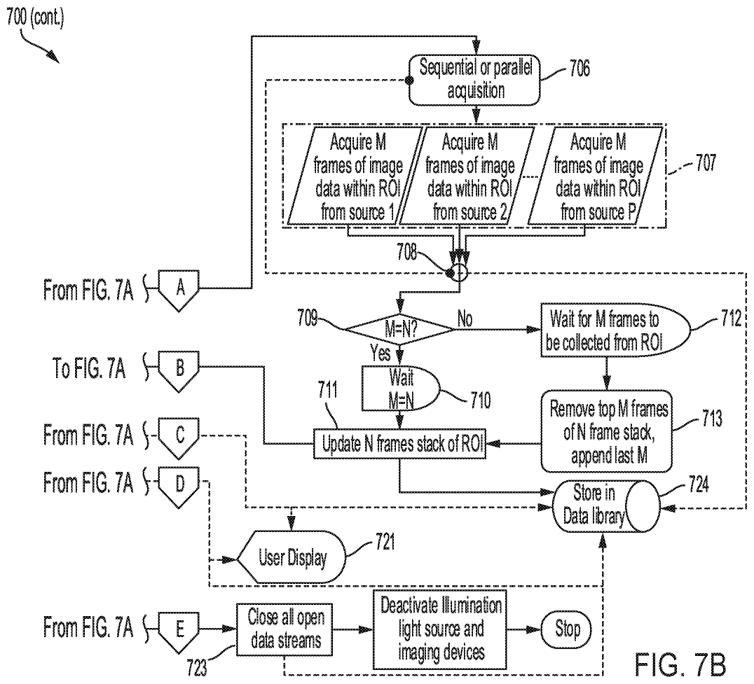

FIGS. 7A and 6B show a flowchart depicting an example embodiment of a process 700 for multi-spectral or multi-wavelength imaging of a target object in the context of laser speckle contrast imaging (LSCI), potentially in combination with other modalities. In the example embodiment, the multi-spectral imaging process 700 can be used for rapid examination of blood hemoglobin oxygenation levels from a FOV, or ROI of the FOV. In various embodiments, the process 700 commences at 701, and awaits a trigger 702 that starts the process 700. The trigger can be manual (i.e., user-generated), automated (i.e., system-generated), or semi-automated (i.e., user- or system-generated). Once triggered 702, the system that implements the multispectral imaging (such as blood oxygen saturation mapping) process 700 proceeds to obtain 703 the parameters, including but not limited to illumination power, duty cycle, time delay, sensor exposure time, frame rate, resolution, binning factor, digitizer gain. The process 700 illuminates the ROI with two or more (or as many as P) illumination sources at 704. Illumination using the two or more illumination sources can be sequential or parallel in terms of activation and/or deactivation, and wavelength or spectral band emitted. In general, for success of multispectral imaging, at least two or more spectral bands undergoing differential absorption within ROI need to be detected separately, in parallel or rapid succession (706 and 708) to avoid motion artifacts or signal perturbation due to dynamic changes within target object. In contrast to the LSCI method, when white or wideband light sources are used, certain excitation selecting and/or emission-passing filters can be set in front of the one or more such sources and/or camera sensors to selectively detect wavelength bands of interest. When narrow wavelength band light sources are used, such as lasers or color LEDs, it may be sufficient to just sequentially turn on and off such sources in sync with frame exposure and acquisition process. Also, here optical system aperture size (if a variable iris is present) can be adjusted to balance sensor sensitivity and imaging depth of view. Some imaging devices also may require application of a dedicated imaging mode or activation of their intensifier or background suppression subsystem and proper timing or delay of all these events. The various parameters can be provided by either the user or obtained from presets in a computer memory. Parameters may be modified manually or automatically using feedback from the imaging result and quality of one or more electronic data registered in real-time during image acquisition. The system then applies these parameters to hardware and then, at 704, activates illumination of the ROI of the target object with an appropriately selected and/or filtered source of light to be partially absorbed by an endogenous or exogenous contrast agent within the tissue; for example, oxy- and deoxyhemoglobins, HbO₂ and Hb, respectively. Once the multispectral image frame set is acquired by the suitably configured acquisition module 120, the information processor module employs rolling FIFO acquisition algorithm, at 705, with the specified number of frames N and at 707, a new set of M frames is acquired and an equivalent number of frames are eliminated from the buffer 713, and updates the N frames stack of the ROI 711. When M=N 709, 710, all N frames in the buffer are replaced with the new set of M frames. Next, this loop restarts and, while waiting 712 for a next set of M frames to arrive, the system employs a subroutine, at 714, to apply one or more of the Image processing algorithms, such as Beer's Law Concentration calculation, contrast enhancement, registration, segmentation, etc., for the pixels of interest in the field of view.

Using the newly arrived data in the stack of N frames residing in buffer, the information processor module generates a monochromatic brightness image, at 714, and also potentially, if overlay is desired 716, computes such quantities as average oxygen saturation level or any other quantity (index) which may be a linear or non-linear function of data acquired, generating a monochrome Image Result 1 at 715. At 717, the system converts Image Result 1 to its pseudo-color representation Image Result 2, according to a user-specified or computer-generated color and brightness table (palette), to provide intuitive and effective visualization of, for example, an oxygen saturation map in vasculature and related characteristics. This visualization can be combined with other imaging modalities. This may necessitate additional processing steps including but not limited to background and offset subtraction, outlier rejection, denoising, bandpass filtering, smoothing and artifact examination of image oxygen saturation map. Process step 718 also implements a subroutine to convert and transform generated image result 717 and other data (from data library 727—comprising of data from other modalities or vital monitoring data, etc.) to compound image data with different data represented in different format thereby presenting more than one image modality data in Image Result 3. The system forwards, at 719, Image Result 2 or Image Result 3, aligning and rescaling 719 as appropriate, depending on the user-selected or preset display setting to AR module 726. In some implementations, the system can utilize techniques to accelerate processing, including parallel computing, analog processing, Machine Vision, Deep Learning, and processing techniques based on estimation rather than exact computation of quantities of interest calculations. In such implementations, post-processing for the stack can be finished before another set of frames is integrated on the imaging device and transferred to computer memory at 708. Based on the parameter settings at 703, the illumination and detection process 700 continues with image acquisition and processing cycles providing real-time examination of target object. In parallel to this process, all the raw data arriving from steps 708, 715, 718 and 723 can be saved to computer random-access memory, or streamed to permanent storage solution or a remote destination for archival and potentially, at 724, more detailed processing and analysis. Based on the parameter settings at 703 or the user input at 720, the imaging process checks to enable or disable, at 719, ARP module 726. Next, at 722, if ‘YES’, the process 700 continues with image acquisition and processing cycle providing real-time examination of chosen ROI. If ‘NO’, the process 700 terminates at 730. During termination of imaging process, 725 switches off the illumination source. In some embodiments, either or both of the Image Result 2 or 3 can be sent to be displayed in external user display devices (TV, monitors, 3D-OLED, etc.) at 721.

Having now described some illustrative embodiments, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one embodiments are not intended to be excluded from a similar role in other embodiments or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate embodiments consisting of the items listed thereafter exclusively. In one embodiment, the systems and methods described herein consist of one, each combination of more than one, or all of the described elements, acts, or components.

Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements to single or plural configurations. References to any act or element being based on any information, act or element may include embodiments where the act or element is based at least in part on any information, act, or element.

Any embodiment disclosed herein may be combined with any other embodiment or embodiment, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one implementation or embodiment. Such terms as used herein are not necessarily all referring to the same embodiment. Any embodiment may be combined with any other embodiment, inclusively or exclusively, in any manner consistent with the aspects and embodiments disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing embodiments are illustrative rather than limiting of the described systems and methods. Scope of the systems and methods described herein is thus indicated by the appended claims, rather than the foregoing description, and changes that come within the meaning and range of equivalency of the claims are embraced therein.

1. A system comprising:
a processor configured to generate a data signal based on image data pertaining to a field of view of a surgical microscope; and
an eyepiece configured to integrate with the surgical microscope and including:
an image generation module configured to generate an image based on the data signal;
an image combiner configured to combine the image generated by the image generation module with light received from the field of view to create a combined image; and
visualization optics configured to present the combined image to an eye of a user of the surgical microscope.

2. The system of claim 1, wherein the data signal includes data pertaining to one or more of images, image sequences, graphical representations, numerical values, or text-based representations.

3. The system of claim 1, comprising:
an image splitter configured to split the light received from the field of view into a first portion and a second portion, the image splitter directing the first portion to the visualization optics; and
a camera module configured to receive the second portion of the light from the image splitter and generate the image data.

4. The system of claim 1, comprising:
an incoherent illumination source; and
illumination optics configured to deliver light from the incoherent illumination source to the field of view.

5. The system of claim 1, comprising:
an illumination module having two or more illumination sources, wherein each illumination source has a narrow wavelength band; and
illumination optics configured to deliver light from the illumination module to the field of view.

6. The system of claim 1, comprising:
a coherent illumination source; and
illumination optics configured to deliver light from the coherent illumination source to the field of view.

7. The system of claim 6, wherein:
the processor is configured to generate laser speckle contrast imaging (LSCI) data based on the image data; and
the data signal includes the LSCI data.

8. The system of claim 1, wherein the processor is configured to generate and display the data signal based on the image data such that the data signal is refreshed faster than a persistence of vision.

9. The system of claim 1, comprising:
a memory storing information relating to the field of view,
wherein the processor is configured to generate the data signal based on the stored information, and provide one or more of a textual, numerical, graphical, or image rendering for display by the image generation module.

10. The system of claim 1, wherein the eyepiece is configured to replace a stock eyepiece of the surgical microscope.

11. A method comprising:
generating a data signal based on image data pertaining to a field of view of a surgical microscope;
generating, using an image generation module of an eyepiece integrated with the surgical microscope, an image based on the data signal;
combining, using an image combiner of the eyepiece, the image with light received from the field of view to create a combined image; and
presenting, using visualization optics of the eyepiece, the combined image to an eye of a user of the surgical microscope.

12. The method of claim 11, wherein the data signal includes data pertaining to one or more of images, image sequences, graphical representations, numerical values, or text-based representations.

13. The method of claim 11, comprising:
splitting the light received from the field of view into a first portion and a second portion;
receiving, at a camera module, the second portion of the light; and
generating, using the camera module, the image data.

14. The method of claim 11, comprising:
illuminating the field of view using an incoherent illumination source.

15. The method of claim 11, comprising:
illuminating the field of view using two or more illumination sources, wherein each illumination source has a narrow wavelength band.

16. The method of claim 11, comprising:
illuminating the field of view using a coherent illumination source.

17. The method of claim 16, comprising:
processing the image data to generate laser speckle contrast imaging (LSCI) data, wherein the data signal includes the LSCI data.

18. The method of claim 11, wherein generating and displaying the data signal based on the image data such that the data signal is refreshed faster than a persistence of vision.

19. The method of any of claim 11, comprising:
storing information relating to the field of view, wherein generating the data signal includes generating the data signal based on the stored information to provide one or more of a textual, numerical, graphical, or image rendering; and
displaying, by the image generation module, the one or more of the textual, numerical, graphical, or image rendering.
